(19) United States
(12) Patent Application Publication
(10) Pub. No.: US 2024/0313733 A1
(43) Pub. Date: Sep. 19, 2024

(54) PHOTOIONIZATION DETECTOR SENSOR WITH ADJUSTABLE GAIN

(71) Applicant: MOCON, INC., Minneapolis, MN (US)

(72) Inventors: Charles Willcox, Hopkins, MN (US); David Jennings, Edina, MN (US); Christopher Fields, Coon Rapids, MN (US)

(21) Appl. No.: 18/184,037

(22) Filed: Mar. 15, 2023

(51) Int. Cl. H03G 3/30

(52) U.S. Cl. CPC H03G 3/3084; H03G 3/3036; H03G 2201/103

(57) ABSTRACT

A photoionization detector sensor equipped with an adjustable gain amplifier, and method of standardizing output of the photoionization detector sensor by adjusting the gain so that an actual test value expected to produce a known anticipated value matches the anticipated value.

### BACKGROUND

Photoionization detector (PID) sensors break molecules, typically volatile organic molecules, into positively charged ions and free electrons using high-energy ultraviolet (UV) radiation. As target analyte enters the detector, they are bombarded by high-energy UV photons. The target analyte absorbs the UV light, resulting in ejection of electrons and the formation of positively charged ions. The positively charged ions are sensed by an anode/cathode arrangement in the detector and produce an electric current whose amplitude is proportional to the positively charged ions produced (i.e., the greater the concentration of target analyte in the sample entering the detector the more positively charged ions produced resulting in a greater current). The current is amplified, displayed on an ammeter, or converted via a look-up table to a concentration of target molecules and digitally displayed.

Manufacturing variances often result in small but meaningful variances in the output of the PID sensors, even amongst those in the same manufactured batch or lot. This requires customers to perform an initial calibration of the PID sensor after placement of the sensor in the customer's instrument.

Hence, a substantial need exists for an OEM PID sensor that eliminates or compensates for manufacturing variances whereby the output of the OEM PID sensor is consistent from sensor to sensor as delivered to customers so as to eliminate the need for customers to perform an initial calibration of the PID sensor after placement of the sensor in the customer's instrument.

### SUMMARY OF THE INVENTION

A first aspect is a photoionization detector sensor equipped with an adjustable gain amplifier. The photoionization detector sensor includes an ultraviolet lamp, electrodes (e.g., an anode-cathode pair), and a signal amplifier. The ultraviolet lamp is operable for ionizing a target analyte within a sample. The electrodes are operable for detecting the presence of ionized target analyte within the sample and generating a current signal proportional to the concentration of target analyte within the sample. The amplifier is in electrical communication with the electrodes for receiving the generated current signal and amplifying the current signal for creating an amplified electrical signal proportional to the concentration of target analyte within the sample. The amplifier includes an adjustable gain feature for allowing the amplified electrical signal output to be standardized to correct for any manufacturing-imposed variance in the current signal generated by the electrodes and transmitted to the amplifier.

A preferred option for the amplifier is a two-stage transimpedance amplifier having an inverting current to voltage first stage and a non-inverting voltage gain second stage wherein the second stage is equipped with a nonvolatile digital potentiometer operable for adjusting the voltage gain.

The photoionization detector sensor can include a housing defining a sample retention chamber operable for holding the sample, the ultraviolet lamp operable for ionizing target analyte within the sample held within the retention chamber, and the electrodes operable for detecting the presence of ionized target analyte within the sample retention chamber.

A second aspect is a method of standardizing output of the photoionization detector sensor in accordance with the first aspect. The method involves the steps of (A) activating the photoionization detector sensor to detect target analyte in a sample to create an amplified electrical signal having a test value, wherein the sample has a known concentration of the target analyte and is expected to generate an amplified electrical signal of known anticipated value, (B) comparing the test value and the anticipated value, and (C) standardizing output of the photoionization detector sensor by adjusting the gain imposed by the amplifier so that the gain-adjusted test value matches the anticipated value.

### BRIEF DESCRIPTION OF THE DRAWINGS

FIG. 1 is a schematic view of one embodiment of the invention.

### DETAILED DESCRIPTION OF A PREFERRED EMBODIMENT

Nomenclature Table

| REF. NO. | DESCRIPTION |
|---|---|
| 100 | Photoionization Detector Sensor |
| 110 | Housing |
| 119 | Sample Retention Chamber |
| 119₁ | Sample Intake Port |
| 119₂ | Sample Venting Port |
| 120 | UV Lamp |
| 130 | Electrodes |
| 130₁ | First Electrode or Anode |
| 130₂ | Second Electrode or Cathode |
| 140 | Amplifier with Adjustable Gain |
| 150 | Processor |
| S₁ | Current Signal from Electrodes |
| S₂ | Amplified and Gain Adjusted Signal from Amplifier |
| A | Target Analyte |

### Construction

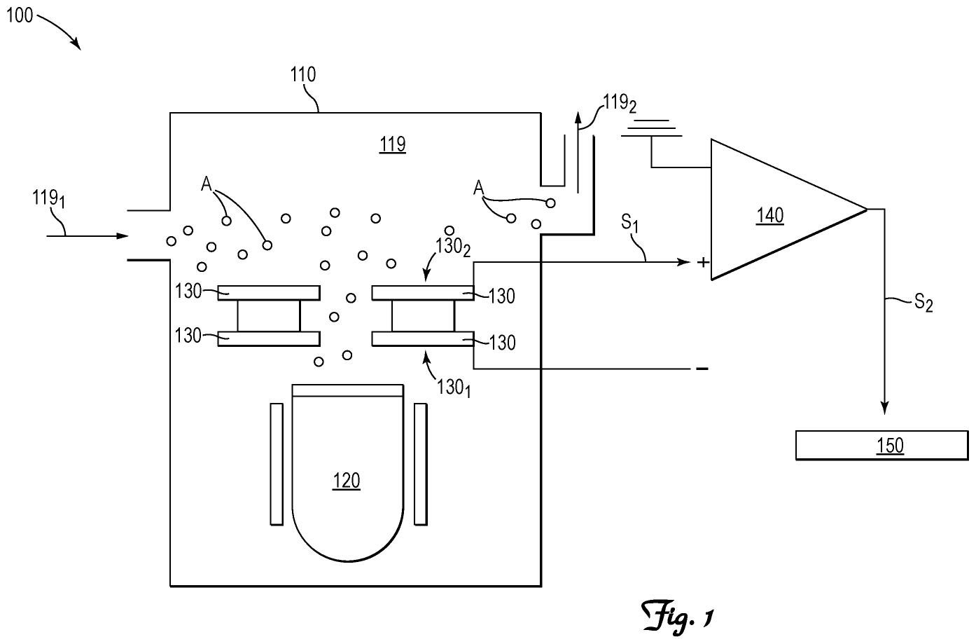

Referring to FIG. 1, the invention is a photoionization detector sensor 100 equipped with an adjustable gain amplifier 140.

Photoionization detector sensors 100 have an ultraviolet (UV) lamp 120 for ionizing target analyte A within a sample, a pair of electrodes 130 (i.e., an anode 130₁ and a cathode 130₂) for detecting the ions and generating a first electrical current signal S₁ proportional to the concentration of target analyte A within the sample, and an amplifier 140 in electrical communication with the electrodes 130 for receiving the generated first electrical current signal S₁ and amplifying and converting the first electrical current signal S₁ to a second voltage electrical signal S₂. These components are generally retained within a housing 110 that defines a sample retention chamber 119 positioned to receive UV radiation emitted by the UV lamp 120 upon excitation of the lamp 120 and having a sample intake port 119₁ and optionally a sample venting port 119₂.

Photoionization detector sensors 100 are employed in instruments that typically include a processor 150 for receiving the amplified electronic signal S₂, converting the value of the amplified electronic signal S₂ to a concentration of target analyte A in the sample based upon an algorithm or a lookup table, and displaying or otherwise reporting the concentration.

The photoionization detector sensor 100 of the present invention has an adjustable gain amplifier 140, such as a two-stage transimpedance amplifier with an inverting current to voltage first stage and a non-inverting voltage gain second stage, wherein the second stage is equipped with a nonvolatile digital potentiometer operable for adjusting the voltage gain.

The photoionization detector sensor 100 is particularly adapted to detect and quantify the concentration of volatile organic compounds in a sample.

### Factory Standardization

The method of standardizing output of the photoionization detector sensor 100 includes the steps of (A) activating the photoionization detector sensor 100 to detect target analyte A in a sample to create an amplified electrical signal S₂ having a test value, wherein the sample has a known concentration of the target analyte A and is expected to generate an amplified electrical signal of known anticipated value, (B) comparing the test value and the anticipated value, and (C) standardizing output of the photoionization detector sensor 100 by adjusting the gain imposed by the amplifier 140 so that the gain-adjusted test value matches the anticipated value.

I claim:

1. A photoionization detector sensor equipped with an adjustable gain amplifier.

2. A photoionization detector sensor having (i) an ultraviolet lamp operable for ionizing a target analyte within a sample, (ii) electrodes for detecting the presence of ionized target analyte within the sample and generating a current signal proportional to the concentration of target analyte within the sample, and (iii) an amplifier in electrical communication with the electrodes for receiving the generated current signal and amplifying the current signal via an adjustable gain to create an amplified electrical signal proportional to the concentration of target analyte within the sample.

3. The photoionization detector sensor of claim 2 wherein the amplifier is a two-stage transimpedance amplifier having an inverting current to voltage first stage and a non-inverting voltage gain second stage wherein the second stage is equipped with a nonvolatile digital potentiometer operable for adjusting the voltage gain.

4. The photoionization detector sensor of claim 2 wherein the ultraviolet lamp is operable for ionizing a volatile organic compound target analyte.

5. A photoionization detector sensor having (i) a housing defining a sample retention chamber, (ii) an ultraviolet lamp operable for ionizing a target analyte within the sample retention chamber, (iii) an anode-cathode pair for detecting the presence of ionized target analyte within the sample retention chamber and generating a current proportional to the concentration of target analyte within the sample retention chamber, and (iv) a two-stage transimpedance amplifier in electrical communication with the anode-cathode pair for receiving the generated current and having an inverting current to voltage first stage to create an electrical signal having a voltage proportional to the concentration of target analyte within the sample, and a non-inverting voltage gain second stage equipped with a nonvolatile digital potentiometer operable for adjusting the voltage gain to create an amplified and adjusted electrical signal proportional to the concentration of target analyte within the sample.

6. The photoionization detector sensor of claim 5 wherein the ultraviolet lamp is operable for ionizing a volatile organic compound target analyte.

7. A method of standardizing output of a photoionization detector sensor in accordance with claim 2, comprising the steps of (A) activating the photoionization detector sensor to detect target analyte in a sample to create an amplified electrical signal having a test value, wherein the sample has a known concentration of the target analyte and is expected to generate an amplified electrical signal of known anticipated value, (B) comparing the test value and the anticipated value, and (C) standardizing output of the photoionization detector sensor by adjusting the gain imposed by the amplifier so that the gain-adjusted test value matches the anticipated value.

8. A method of standardizing output of a photoionization detector sensor in accordance with claim 5, comprising the steps of (A) activating the photoionization detector sensor to detect target analyte in a sample to create an amplified electrical signal having a test value, wherein the sample has a known concentration of the target analyte and is expected to generate an amplified electrical signal of known anticipated value, (B) comparing the test value and the anticipated value, and (C) standardizing output of the photoionization detector sensor by adjusting the gain imposed by the non-inverting voltage gain second stage of the two-stage transimpedance amplifier so that the gain-adjusted test value matches the anticipated value.
